(19) United States
(12) Patent Application Publication
(10) Pub. No.: US 2026/0165917 A1
(43) Pub. Date: Jun. 18, 2026

(54) NON-STICKY STABLE COMPOSITION WITH TEXTURE TRANSFORMATION PROPERTY

(71) Applicant: L'OREAL, Paris (FR)

(72) Inventors: Toru KOIKE, Kawasaki-shi (JP); Kazuhiko MARUYAMA, Kawasaki-shi (JP); Tomomi HAMAZAKI, Kawasaki-shi (JP)

(21) Appl. No.: 19/129,464

(22) PCT Filed: Nov. 10, 2023
(86) PCT No.: PCT/JP2023/041376

§ 371 Date: May 13, 2025

(30) Foreign Application Priority Data
Nov. 18, 2022 (JP) 2022-185108
Nov. 18, 2022 (JP) 2022-185109
Jan. 19, 2023 (FR) 2300489
Jan. 19, 2023 (FR) 2300499

(51) Int. Cl. A61K 8/06; A61K 8/73; A61K 8/81; A61Q 17/04

(52) U.S. Cl. CPC A61K 8/062; A61K 8/731; A61K 8/817; A61Q 17/04; A61K 2800/413; A61K 2800/48

(57) ABSTRACT

A composition contains: (a) at least one cellulose particle; (b) at least one associative polymer comprising one or more acrylic and/or methacrylic units; and (c) water. According to one embodiment, the composition is stable and can provide no sticky feeling or a reduced sticky feeling, after application, in addition to texture transformation during application.

### TECHNICAL FIELD

The present invention relates to a composition, preferably a cosmetic composition, and more preferably a skin cosmetic composition, which is stable and can provide texture transformation and non-stickiness.

### BACKGROUND ART

Imparting a comfortable texture to skin is one of the key features of cosmetic products, in particular skincare cosmetic products.

Especially, texture transformation (change in texture from, for example, a gel or cream to a liquid, which corresponds to the change in physical properties of a composition, such as a collapse of the structure of the composition which could allow liquid to flow out from the composition) which could provide a refreshing feel at the early stage of application, as well as a non-sticky feeling or a reduced sticky feeling after application, are still needed for consumer satisfaction on use.

It should be noted that a composition which is unstable and easily causes a collapse of the structure of the composition often provides a comfortable feel on use. This means that it is difficult to provide both a comfortable feel on use and good stability at the same time.

So far, several technologies regarding gel-like compositions or emulsion compositions which impart an improved texture and stability have been reported. For example, WO2022/124385 discloses a stable composition which can provide texture transformation.

### DISCLOSURE OF INVENTION

However, sufficient investigations have not yet been made for stable compositions which can provide no or reduced sticky feeling, in addition to texture transformation.

An objective of the present invention is to provide a stable composition which can provide no or reduced sticky feeling, after application, in addition to texture transformation during application.

The above objective of the present invention can be achieved by a composition, preferably a cosmetic composition, and more preferably a skin cosmetic composition, comprising:
(a) at least one cellulose particle;
(b) at least one associative polymer comprising one or more acrylic and/or methacrylic units; and
(c) water.

The particle size of the (a) cellulose particle may be 50 μm or less, preferably 30 μm or less, and more preferably 10 μm or less.

The (a) cellulose particle may have
a wet point for oil of at least 40 ml/100 g, preferably at least 50 ml/100 g, and more preferably at least 60 ml/100 g, and
a wet point for water of at least 100 ml/100 g, preferably at least 200 ml/100 g, and more preferably at least 300 ml/100 g.

The ratio of the wet point for water/the wet point for oil of the (a) cellulose particle may be 5 or less, preferably 4 or less, and more preferably 2 or less.

The (a) cellulose particle may be spherical.

The amount of the (a) cellulose particle(s) in the composition according to the present invention may be from 0.1% to 20% by weight, preferably from 0.5% to 15% by weight, and more preferably from 1% to 10% by weight relative to the total weight of the composition.

The (b) associative polymer may comprise one or more (meth)acrylic acid (C₁-C₁₂)alkyl ester units.

The (b) associative polymer may comprise one or more units derived from polyoxyalkylenated fatty alcohols.

The amount of the (b) associative polymer(s) in the composition according to the present invention may be from 0.01% to 10% by weight, preferably from 0.05% to 5% by weight, and more preferably from 0.1% to 1% by weight relative to the total weight of the composition.

The composition according to the present invention may further comprise (d) at least one lipophilic organic UV filter.

The (d) lipophilic organic UV filter may be selected from the group consisting of drometrizole trisiloxane, bis(ethylhexyloxyphenol)methoxyphenyltriazine, ethylhexyl triazone, diethylamino hydroxybenzoyl hexyl benzoate, butyl methoxybenzoylmethane, ethylhexyl methoxycinnamate, ethylhexyl salicylate, octocrylene, homosalate, and a mixture thereof.

The amount of the (d) lipophilic organic UV filter(s) in the composition according to the present invention may be from 1% to 30% by weight, preferably from 3% to 25% by weight, and more preferably from 5% to 20% by weight relative to the total weight of the composition.

The composition according to the present invention may further comprise (e) at least one oil, preferably selected from polar oils, and more preferably selected from ester oils, artificial triglycerides, and mixtures thereof.

The composition according to the present invention may further comprise (f) at least one nonionic surfactant, preferably selected from polyglyceryl fatty acid esters, and more preferably polyglyceryl fatty acid monoesters.

The present invention also relates to a cosmetic process for treating a keratin substance, comprising the step of applying the composition according to the present invention to the keratin substance.

### BEST MODE FOR CARRYING OUT THE INVENTION

After diligent research, the inventors have discovered a new approach to provide a stable composition which can provide no or reduced sticky feeling, after application, in addition to texture transformation during application.

Thus, one aspect of the present invention is a composition comprising:
(a) at least one cellulose particle;
(b) at least one associative polymer comprising one or more acrylic and/or methacrylic units; and
(c) water.

The composition according to the present invention is stable and can provide no or reduced sticky feeling, after application, in addition to texture transformation during application.

The composition according to the present invention is stable just after the preparation of the composition and for a long time after the preparation of the composition, even under temperature changes from a cold to hot temperature. Therefore, the composition according to the present invention is stable over time, and can be stored for a long period of time even under temperature changes occurring from winter to summer.

The composition according to the present invention can provide no sticky feeling or a reduced sticky feeling, after application.

The term “sticky” here means a property which provides a tacky feeling to the skin.

The composition according to the present invention can provide texture transformation during application, preferably at the early stage of application. The texture transformation here means a change in texture from, for example, a gel or cream to a liquid, which corresponds to the change in physical properties of a composition, such as a collapse of the structure of the composition which could allow liquid to flow out from the composition. The texture transformation can provide refreshing feeling.

Therefore, the composition according to the present invention can provide an excellent feeling to touch, in particular on feeling of the skin during and after application of the composition.

Even if the composition according to the present invention comprises (d) at least one lipophilic organic UV filter, which has properties similar to those of oil and may cause oily or greasy feeling and shiny aspect such as shiny skin, after application, the composition according to the present invention can provide no or reduced oily or greasy feeling, and no or reduced shiny aspect, such as shiny skin

Thus, even if the composition according to the present invention comprises (d) at least one lipophilic organic UV filter, the composition according to the present invention is stable and can provide no or reduced sticky feeling, as well as no or reduced oily/greasy feeling, and no or reduced shiny aspect, after application, in addition to texture transformation during application.

Hereinafter, the composition according to the present invention and the like will be explained in a more detailed manner.

### [Cellulose Particle]

The composition according to the present invention includes (a) at least one cellulose particle with specific properties. If two or more cellulose particles are used, they may be the same or different.

The particle size of the (a) cellulose particle corresponds to a primary particle size. In addition, the particle size here means an average or mean particle size, which may be a volume-average particle size, which can be determined by, for example, a laser diffraction particle size analyzer.

The particle size of the (a) cellulose particle used for the present invention is not limited. However, it is preferable that the particle size of the (a) cellulose particle be 50 μm or less, preferably 30 μm or less, more preferably 15 μm or less, even more preferably 10 μm or less, and particularly preferably from 2 to 5 μm.

It is preferable that 90 vol % or more of the (a) cellulose particles used for the present invention have an average primary particle size ranging from 0.1 to 10 μm, preferably from 0.5 to 8 μm, and more preferably from 1 to 7 μm. If 90 vol % or more of the (a) cellulose particles have an average primary particle size ranging from 1 to 7 μm, optical effects due to the particles may also be achieved.

The (a) cellulose particle used for the present invention may be in any particulate form.

It is preferable that the ratio of the longest diameter/the shortest diameter of the (a) cellulose particle used for the present invention range from 1.0 to 10, preferably from 1.0 to 5, and more preferably from 1.0 to 3.

It is more preferable that the (a) cellulose particle be spherical, preferably with a ratio of the longest diameter/the shortest diameter of from 1.0 to 1.1.

The (a) cellulose particle used for the present invention may have
a wet point for oil being at least 40 ml/100 g, preferably at least 50 ml/100 g, more preferably at 30 least 60 ml/100 g, more preferably at least 100 ml/100 g, and even more preferably at least 250 ml/100 g, and preferably 1500 ml/100 g or less; and
a wet point for water being at least 100 ml/100 g, preferably at least 200 ml/100 g, more preferably at least 300 ml/100 g, even more preferably at least 350 ml/100 g, and preferably 1500 ml/100 g or less.

The term “wet point for oil” in the specification means a quantity or amount of oil which is necessary to make a target powder completely wet, which can be recognized, in particular, by the formation of a paste with the target powder.

The wet point for oil can be determined by the following protocol.
(1) 2 g of a target powder is kneaded with a spatula on a glass plate while adding oil, in particular linear ester oil, such as isononyl isononanoate (WICKENOL 151/ALZO).
(2) When the target powder becomes completely wet and starts to form a paste, the weight of the added oil is determined as the weight of the wet point.
(3) The wet point for oil is calculated from the equation: Wet point for oil (ml/100 g)={(the weight of the wet point)/2 g}×100/the density of oil.

Similarly, the term “wet point for water” in the specification means a quantity or amount of water which is necessary to make a target powder completely wet, which can be recognized, in particular, by the formation of a paste with the target powder.

The wet point for water can be determined by the following protocol.
(1) 2 g of a target powder is kneaded with a spatula on a glass plate while adding water with a density of 0.998 g/ml.
(2) When the target powder becomes completely wet and starts to form a paste, the weight of the added water is determined as the weight of the wet point.
(3) The wet point for water is calculated from the equation: Wet point for water (ml/100 g)={(the weight of the wet point)/2 g}×100/the density of water.

It is preferable that the ratio of the wet point for water/the wet point for oil of the (a) cellulose particle used for the present invention be 5 or less, preferably 4 or less, more preferably 3 or less, and even more preferably 2 or less, and preferably 0.1 or more.

The (a) cellulose particle may be porous or non-porous, preferably porous.

The porosity of the (a) cellulose particle may be characterized by a specific surface area of from 0.05 m²/g to 1,500 m²/g, more preferably from 0.1 m²/g to 1,000 m²/g, and even more preferably from 0.2 m²/g to 500 m²/g according to the BET method.

In the present invention, the cellulose that may be used for the (a) cellulose particle is not limited by the types of cellulose such as cellulose I, cellulose II, or the like. As the cellulose which can be used as a material for the (a) cellulose particle for the present invention, type II cellulose is preferable.

The (a) cellulose particle, preferably a spherical cellulose particle, can be prepared, for example, as follows.
(1) A slurry of calcium carbonate, as an aggregation inhibitor, is added to an alkaline water-soluble anionic polymer aqueous solution, and stirred,
(2) Viscose and the aqueous solution obtained in the above (1) are mixed to form a dispersion of viscose fine particles.
(3) The dispersion of viscose fine particles obtained in the above (2) is heated to aggregate the viscose in the dispersion, and neutralized with acid, to form cellulose fine particles.
(4) The cellulose fine particles are separated from the mother liquid obtained in the above (3), and washed and dried, if necessary.

The viscose is a raw material of the cellulose. It is preferable to use viscose with a gamma value of 30 to 100% by mass and an alkaline concentration of 4 to 10% by mass. As the above water-soluble anionic polymer, mention may be made of polyacrylic acid sodium salt, polystyrene sulfonic acid sodium salt, and the like. The above calcium carbonate is used to prevent the aggregation of viscose fine particles in the dispersion and to make the particle size of the cellulose particle smaller. As the calcium carbonate slurry, mention may be made of Tama Pearl TP-221GS marketed by Okutama Kogyo Co., Ltd. in Japan.

According to one embodiment, the (a) cellulose particle may or may not include a cellulose derivative. The cellulose derivative may be chosen from cellulose esters and ethers.

It is indicated that the term “cellulose ester” means, in the text hereinabove and hereinbelow, a polymer consisting of an α (1-4) sequence of partially or totally esterified anhydroglucose rings, the esterification being obtained by the reaction of all or only some of the free hydroxyl functions of the said anhydroglucose rings with a linear or branched carboxylic acid or carboxylic acid derivative (acid chloride or acid anhydride) containing from 1 to 4 carbon atoms.

Preferably, the cellulose ester results from the reaction of some of the free hydroxyl functions of said rings with a carboxylic acid containing from 1 to 4 carbon atoms.

Advantageously, the cellulose esters are chosen from cellulose acetates, propionates, butyrates, isobutyrates, acetobutyrates and acetopropionates, and mixtures thereof.

These cellulose esters may have a weight-average molecular mass ranging from 3,000 to 1,000,000, preferably from 10,000 to 500,000 and more preferably from 15,000 to 300,000.

In the text hereinabove and hereinbelow, the term “cellulose ether” means a polymer consisting of an α (1-4) sequence of partially etherified anhydroglucose rings, some of the free hydroxyl functions of said rings being substituted with a radical —OR, R preferably being a linear or branched alkyl radical containing from 1 to 4 carbon atoms.

The cellulose ethers are thus preferably chosen from cellulose alkyl ethers with an alkyl group containing from 1 to 4 carbon atoms, such as cellulose methyl, propyl, isopropyl, butyl and isobutyl ethers.

These cellulose ethers may have a weight-average molecular mass ranging from 3,000 to 1,000,000, preferably from 10,000 to 500,000 and more preferably from 15,000 to 300,000.

As the (a) cellulose particle used for the present invention, mention may be made of, for example, the following spherical cellulose particles marketed by Daito Kasei in Japan:
Cellulobeads USF (the wet point for oil is 296.0 ml/100 g, the wet point for water is 400.8 ml/100 g, and the ratio of the wet point for water/the wet point for oil is 1.4) with an average particle size of 4 μm;
Cellulobeads USF-X (the wet point for oil is, at maximum, 80 ml/100 g, the wet point for water is 250 ml/100 g, and the ratio of the wet point for water/the wet point for oil is 3.1 or less) with an average particle size of 3-5 μm;
Cellulobeads D-5 (the wet point for oil is, at maximum, 70 ml/100 g, the wet point for water is 150 ml/100 g, and the ratio of the wet point for water/the wet point for oil is 2.1 or less) with an average particle size of 8-10 μm; and
Cellulobeads D-10 (the wet point for oil is, at maximum, 60 ml/100 g, the wet point for water is 140 ml/100 g, and the ratio of the wet point for water/the wet point for oil is 2.3 or less) with an average particle size of 12-15 μm.

The (a) cellulose particle used for the present invention may or may not be coated beforehand.

In a particular embodiment, the (a) cellulose particle is originally coated. The material of an original coating of the particle is not limited, but an organic material such as a mono- or di-carboxylic acid or a salt thereof, an amino acid, an N-acylamino acid, an amido, a silicone and a modified silicone may be preferable. As the organic material, mention may be made of potassium succinate, lauroyl lysine and acryl-modified silicone.

In other words, the (a) cellulose particle used for the present invention may be surface-treated. As examples of the surface treatments, mention may be made of the following:
(1) Fluorine-based compound treatments such as treatments with perfluoroalkylphosphates, perfluoroalkylsilanes, perfluoropolyethers, fluorosilicones, and fluorinated silicone resins
(2) Silicone treatments such as treatments with methylhydrogenpolysiloxanes, dimethylpolysiloxanes, and tetramethyltetrahydrogencyclotetrasiloxane in a gas phase
(3) Pendant treatments such as treatments to add an alkyl chain and the like after the gas phase silicone treatment
(4) Silane coupling agent treatments
(5) Titanium coupling agent treatments
(6) Aluminum coupling agent treatments
(7) Oil agent treatments
(8) N-acylated lysine treatments
(9) Polyacrylic acid treatments
(10) Metal soap treatments such as those with stearate salt or myristate salt
(11) Acrylic resin treatments
(12) Metal oxide treatments

It is possible to perform a plurality of surface treatments in combination with the above treatments.

The amount of the (a) cellulose particle(s) in the composition according to the present invention may be from 0.1% by weight or more, preferably 0.5% by weight or more, and more preferably 1% by weight or more, relative to the total weight of the composition.

The amount of the (a) cellulose particle(s) in the composition according to the present invention may be 20% by weight or less, preferably 15% by weight or less, and more preferably 10% by weight or less, relative to the total weight of the composition.

The amount of the (a) cellulose particle(s) in the composition according to the present invention may be from 0.1% to 20% by weight, preferably from 0.5% to 15% by weight, and more preferably from 1% to 10% by weight relative to the total weight of the composition.

### [Associative Polymer]

The composition according to the present invention comprises (b) at least one associative polymer comprising one or more acrylic and/or methacrylic units. If two or more such associative polymers are used, they may be the same or different.

The (b) associative polymer can function as a thickener.

For the purpose of the present invention, the term “associative polymer” refers to homopolymers or copolymers that are capable, in an aqueous medium, of reversibly combining with each other or with other molecules.

The (b) associative polymer used for the present invention is preferably an associative copolymer of one or more acrylic and/or methacrylic units and of other units.

The (b) associative polymer more particularly comprises at least one hydrophilic part and at least one hydrophobic part. For the purpose of the present invention, the term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 16 to 30 carbon atoms.

For the purpose of the present invention, the (b) associative polymer comprising one or more acrylic and/or methacrylic units means that the polymer comprises at least one acrylic acid unit or at least one methacrylic acid unit or a mixture thereof. The (b) associative polymer may comprise further units such as units formed by an alkyl ester of acrylic acid or methacrylic acid, preferably of acrylic acid, comprising less than 6 carbon atoms: for example, a C₁-C₄ alkylacrylate, for example, chosen from methyl acrylate, ethyl acrylate and butyl acrylate, called hereinafter “simple ester”.

In one preferred embodiment of the present invention, the (b) associative polymer used for the present invention comprises one or more (meth)acrylic acid (C₁-C₁₂) alkyl ester units, more preferably one or more (meth)acrylic acid (C₁-C₆) alkyl ester units.

According to a particular embodiment, the (b) associative polymer used for the present invention comprises one or more acrylic acid units. According to a further embodiment, the (b) associative polymer used for the present invention comprises one or more methacrylic acid units. According to another embodiment, the (b) associative polymer used for the present invention comprises one or more acrylic acid units and one or more methacrylic acid units.

In a particular embodiment, the (b) associative polymer may comprise another unit derived from another monomer that is different from (meth)acrylic acid. For example, such monomer may be esters of ethylenically unsaturated hydrophilic monomers, such as (meth)acrylic acid or itaconic acid, and of polyoxyalkylenated fatty alcohols. Preferably, the fatty alcohol moiety of the ester monomer can be linear or branched, preferably linear, saturated or unsaturated, preferably saturated, (C₁₂-C₃₀) fatty alcohol, and in particular (C₁₆-C₂₆) fatty alcohol. The polyoxyalkylene chain of the ester monomer preferentially consists of ethylene oxide units and/or propylene oxide units and even more particularly consists of ethylene oxide units. The number of oxyalkylene units generally ranges from 3 to 100, preferably from 7 to 50 and more preferably from 12 to 30.

As examples of the (b) associative polymer, mention may be made, for example, of the product sold under the commercial name: NOVETHIX L-10 POLYMER® (INCI name: ACRYLATES/BEHENETH-25 METHACRYLATE COPOLYMER) sold by LUBRIZOL, the product under the commercial name: Aculyn®28 (INCI name: ACRYLATES/BEHENETH-25 METHACRYLATE COPOLYMER) sold by DOW CHEMICAL, the product sold under the commercial name: Aculyn®22 (INCI name: ACRYLATES/STEARETH-20 METHACRYLATE COPOLYMER) sold by DOW CHEMICAL, the product sold under the commercial name: Aculyn® 88 (INCI name: ACRYLATES/STEARETH-20 METHACRYLATE CROSSPOLYMER) sold by DOW CHEMICAL, the product sold under the commercial name: STRUCTURE® 2001 (INCI name: ACRYLATES/STEARETH-20 ITACONATE COPOLYMER) sold by AKZO NOBEL, and the product sold under the commercial name: STRUCTURE® 3001 (INCI name: ACRYLATES/CETETH-20 ITACONATE COPOLYMER) sold by AKZO NOBEL.

The (b) associative polymer that can be used for the present invention may also be chosen from anionic associative (co)polymers containing acrylic and/or methacrylic units.

The (meth)acrylic anionic associative (co)polymer that may be used for the present invention may be chosen from those comprising at least one hydrophilic unit of unsaturated olefinic carboxylic acid type, and at least one hydrophobic unit of a type such as a (C₁₀-C₃₀) alkyl ester of an unsaturated carboxylic acid.

More particularly, these (meth)acrylic associative (co)polymers are preferably chosen from those in which the hydrophilic unit of unsaturated olefinic carboxylic acid type corresponds to the monomer of formula (I) below:

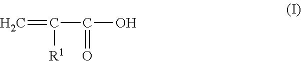

in which R¹ denotes H or CH₃, i.e. acrylic acid or methacrylic acid units, and in which the hydrophobic unit of (C₁₀-C₃₀) alkyl ester of unsaturated carboxylic acid type corresponds to the monomer of formula (II) below:

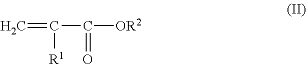

in which R¹ denotes H or CH₃ (i.e. acrylate or methacrylate units), R² denoting a C₁₀-C₃₀ and preferably a C₁₂-C₂₂ alkyl radical.

As (C₁₀-C₃₀)alkyl esters of unsaturated carboxylic acids according to formula (II), mention may more particularly be made of lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

(Meth)acrylic associative (co)polymers of this type are described and prepared, for example, according to U.S. Pat. Nos. 3,915,921 and 4,509,949.

The (meth)acrylic associative (co)polymer that may be used for the present invention may more particularly denote polymers formed from a mixture of monomers comprising:
(i) acrylic acid and one or more esters of formula (III) below:

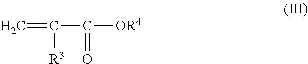

in which R³ denotes H or CH₃, R⁴ denoting an alkyl radical having from 12 to 22 carbon atoms, and optionally a crosslinking agent, for instance those consisting of from 95% to 60% by weight of acrylic acid (hydrophilic unit), 4% to 40% by weight of C₁₀-C₃₀ alkyl acrylate (hydrophobic unit), and 0 to 6% by weight of crosslinking polymerizable monomer, or 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer; or
(ii) essentially acrylic acid and lauryl methacrylate, such as the product formed from 66% by weight of acrylic acid and 34% by weight of lauryl methacrylate.

For these embodiments of the present invention, the term “crosslinking agent” can mean a monomer containing the group

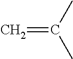

and at least one other polymerizable group, the unsaturated bonds of the monomer being unconjugated relative to each other. As the crosslinking agent that may be used for the present invention, mention may especially be made of polyallyl ethers especially such as polyallyl sucrose and polyallylpentaerythritol.

Among the said (meth)acrylic associative (co)polymers above, the ones particularly preferred for the present invention are the products sold by the company Goodrich under the trade names Pemulen TR1, Pemulen TR2, Carbopol 1382, and more preferably still Pemulen TR1, and the product sold by the company S.E.P.C. under the name Coatex SX.

As the (meth)acrylic associative (co)polymer, mention may also be made of the copolymer of methacrylic acid/methyl acrylate/dimethyl-meta-isopropenylbenzyl isocyanate of ethoxylated alcohol sold under the name Viscophobe DB 1000 by the company Amerchol.

Other (meth)acrylic associative (co)polymers that may be used for the present invention may also be sulfonic polymers comprising at least one (meth)acrylic monomer bearing sulfonic group(s), in free form or partially or totally neutralized form and comprising at least one hydrophobic portion.

The said hydrophobic portion present in the said sulfonic polymers that may be used for the present invention preferably comprises from 8 to 22 carbon atoms, more preferably from 8 to 18 carbon atoms and more particularly from 12 to 18 carbon atoms.

Preferentially, these sulfonic polymers that may be used for the present invention are partially or totally neutralized with a mineral base (sodium hydroxide, potassium hydroxide or aqueous ammonia) or an organic base such as mono-, di- or triethanolamine, an aminomethylpropanediol, N-methylglucamine, basic amino acids, for instance arginine and lysine, and mixtures of these compounds.

These said sulfonic polymers generally have a number-average molecular weight ranging from 1000 to 20 000 000 g/mol, preferably ranging from 20 000 to 5 000 000 g/mol and even more preferably from 100 000 to 1 500 000 g/mol.

The sulfonic polymers that may be used for the present invention may or may not be crosslinked. Crosslinked polymers are preferably chosen.

When they are crosslinked, the crosslinking agents may be selected from polyolefinically unsaturated compounds commonly used for the crosslinking of polymers obtained by free-radical polymerization. Mention may be made, for example, of divinylbenzene, diallyl ether, dipropylene glycol diallyl ether, polyglycol diallyl ethers, triethylene glycol divinyl ether, hydroquinone diallyl ether, ethylene glycol diacrylatedi(meth)acrylate or tetraethylene glycol diacrylatedi(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

Methylenebisacrylamide, allyl methacrylate or trimethylolpropane triacrylate (TMPTA) will be used more particularly.

The degree of crosslinking will generally range from 0.01 mol % to 10 mol % and more particularly from 0.2 mol % to 2 mol % relative to the polymer.

The (meth)acrylic monomers bearing sulfonic group(s) of the sulfonic polymers that may be used for the present invention are chosen especially from (meth)acrylamido(C₁-C₂₂)alkylsulfonic acids and N—(C₁-C₂₂)alkyl(meth)acrylamido(C₁-C₂₂)alkylsulfonic acids, for instance undecylacrylamidomethanesulfonic acid, and also partially or totally neutralized forms thereof. (Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used. 2-Acrylamido-2-methylpropanesulfonic acid (AMPS), and also partially or totally neutralized forms thereof, will even more particularly be used.

The (meth)acrylic associative thickeners that may be used for the present invention may be chosen especially from random amphiphilic AMPS polymers modified by reaction with a C₆-C₂₂ n-monoalkylamine or C₆-C₂₂ di-n-alkylamine, such as those described in patent application WO 00/31154 (which forms an integral part of the content of the description).

These polymers may also contain other ethylenically unsaturated hydrophilic monomers selected, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The (meth)acrylic associative thickeners bearing sulfonic group(s) that may particularly preferably be used for the present invention are preferably chosen from amphiphilic copolymers of AMPS and of at least one ethylenically unsaturated hydrophobic monomer comprising at least one hydrophobic portion containing from 8 to 50 carbon atoms, more preferably from 8 to 22 carbon atoms, more preferably still from 8 to 18 carbon atoms and more particularly 12 to 18 carbon atoms.

These same copolymers may also contain one or more ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described especially in patent application EP-A-750 899, U.S. Pat. No. 5,089,578 and in the following Yotaro Morishima publications:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, 2000, Vol. 33, No. 10-3694-3704;
Solution properties of micelle networks formed by nonionic moieties covalently bound to a polyelectrolyte: salt effects on rheological behavior—Langmuir, 2000, Vol. 16, No. 12, 5324-5332;
Stimuli responsive amphiphilic copolymers of sodium 2-(acrylamido)-2-methylpropanesulfonate and associative macromonomers, Polym. Preprint, Div. Polym. Chem. 1999, 40(2), 220-221.

The ethylenically unsaturated hydrophobic monomers of these particular copolymers are preferably selected from the acrylates or acrylamides of formula (IV) below:

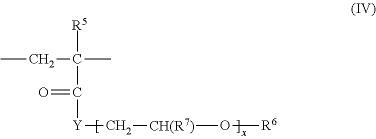

in which R⁵ and R⁷, which may be identical or different, denote a hydrogen atom or a linear or branched C₁-C₆ alkyl radical (preferably methyl); Y denotes O or NH; R⁶ denotes a hydrophobic hydrocarbon-based radical containing at least 8 to 50 carbon atoms, more preferentially from 8 to 22 carbon atoms, even more preferentially from 6 to 18 carbon atoms and more particularly from 12 to 18 carbon atoms; x denotes the number of moles of alkylene oxide and ranges from 0 to 100.

The radical R⁶ is preferably chosen from linear C₆-C₁₈ alkyl radicals (for example n-hexyl, n-octyl, n-decyl, n-hexadecyl, n-dodecyl), or branched or cyclic C₆-C₁₈ alkyl radicals (for example cyclododecane (C₁₂) or adamantane (C₁₀)); C₆-C₁₈ perfluoroalkyl radicals (for example the group of formula —(CH₂)₂—(CF₂)₉—CF₃); the cholesteryl radical (C₂₇) or a cholesterol ester residue, for instance the cholesteryl oxyhexanoate group; aromatic polycyclic groups such as naphthalene or pyrene. Among these radicals, the ones that are more particularly preferred are linear alkyl radicals and more particularly the n-dodecyl radical.

According to a particularly preferred form of the present invention, the monomer of formula (IV) comprises at least one alkylene oxide unit (x≥1) and preferably a polyoxyalkylene chain. The polyoxyalkylene chain preferentially consists of ethylene oxide units and/or propylene oxide units and even more particularly consists of ethylene oxide units. The number of oxyalkylene units generally ranges from 3 to 100, more preferably from 3 to 50 and more preferably still from 7 to 25.

Among these polymers, mention may be made of:
copolymers, which may or may not be crosslinked and which may or may not be neutralized, comprising from 15% to 60% by weight of AMPS units and from 40% to 85% by weight of (C₈-C₁₆)alkyl(meth)acrylamide units or of (C₈-C₁₆)alkyl (meth)acrylate units, relative to the polymer, such as those described in patent application EP-A-750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Mention will be made of the copolymers constituted of 2-acrylamido-2-methylpropanesulfonic acid (AMPS) units of formula (V) below:

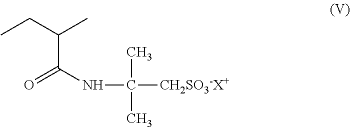

in which X⁺ is a proton, an alkali metal cation, an alkaline-earth metal cation or an ammonium ion;
and units of formula (VI) below:

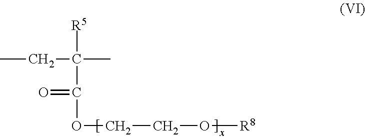

in which x denotes an integer ranging from 3 to 100, preferably from 5 to 80 and more preferentially from 7 to 25; R⁵ has the same meaning as that indicated above in formula (IV) and R⁸ denotes a linear or branched C₆-C₂₂ and more preferentially C₁₀-C₂₂ alkyl.

The polymers that are preferred are those for which x=25, R⁵ denotes methyl and R⁸ represents n-dodecyl; they are described in the Morishima articles mentioned above.

Furthermore, in formula (IV), the use of the monomers where x=20 to 25, R⁵ denotes methyl and R⁸ represents a C₁₂-C₂₄ alkyl group, preferably, for example, a lauryl group, myristyl group, palmityl group, stearyl group, or behenyl group is particularly preferred. Especially, the use of the monomers of the formula (IV), wherein x=20 to 25, R⁵ denotes methyl and R⁸ represents a C₁₆-C₂₄ alkyl chain, such as a stearyl group, or behenyl group is particularly preferred.

The polymers for which X* denotes sodium or ammonium are more particularly preferred.

The amount of the (b) associative polymer(s) in the composition according to the present invention may be from 0.01% by weight or more, preferably 0.05% by weight or more, and more preferably 0.1% by weight or more, relative to the total weight of the composition.

The amount of the (b) associative polymer(s) in the composition according to the present invention may be 10% by weight or less, preferably 5% by weight or less, and more preferably 1% by weight or less, relative to the total weight of the composition.

The amount of the (b) associative polymer(s) in the composition according to the present invention may be from 0.01% to 10% by weight, preferably from 0.05% to 5% by weight, and more preferably from 0.1% to 1% by weight relative to the total weight of the composition.

### [Water]

The composition according to the present invention comprises (c) water.

The amount of (c) water in the composition according to the present invention may be 25% by weight or more, preferably 30% by weight or more, and more preferably 35% by weight or more, relative to the total weight of the composition.

On the other hand, the amount of (c) water in the composition according to the present invention may be 95% by weight or less, preferably 90% by weight or less, and more preferably 85% by weight or less, relative to the total weight of the composition.

The amount of (c) water in the composition according to the present invention may range from 25% to 95% by weight, preferably from 30% to 90% by weight, more preferably from 35% to 85% by weight, relative to the total weight of the composition.

### [Lipophilic Organic UV Filter]

The composition according to the present invention may comprise (d) at least one lipophilic organic UV filter, A single type of lipophilic organic UV filter may be used, but two or more different types of lipophilic organic UV filter may be used in combination.

The term “lipophilic organic UV filter” means an organic molecule that is capable of screening out UV radiation, preferably between 290 and 400 nm, and which can be dissolved in or dispersed in a fatty phase, including a fatty substance such as an oil, in order to obtain a macroscopically homogeneous phase. The term “organic molecule” means any molecule comprising in its structure one or more carbon atoms. Thus, the (d) lipophilic organic UV filter used for the present invention may be active in the UV-A and/or UV-B region.

The (d) lipophilic organic UV filter may be solid or liquid. The terms “solid” and “liquid” mean solid and liquid, respectively, at 25° C. under 1 atm.

The (d) lipophilic organic UV filter may be chosen especially from cinnamic derivatives; anthranilates; salicylic derivatives; dibenzoylmethane derivatives, camphor derivatives; benzophenone derivatives; β,β-diphenylacrylate derivatives; triazine derivatives; benzotriazole derivatives; benzalmalonate derivatives, especially those mentioned in U.S. Pat. No. 5,624,663; imidazolines; p-aminobenzoic acid (PABA) derivatives; benzoxazole derivatives as described in patent applications EP 0 832 642, EP 1 027 883, EP 1 300 137, and DE 101 62 844; screening polymers and screening silicones such as those described especially in patent application WO 93/04665; α-alkylstyrene-based dimers, such as those described in patent application DE 198 55 649; 4,4-diarylbutadienes such as those described in patent applications EP 0 967 200, DE 197 46 654, DE 197 55 649, EP-A-1 008 586, EP 1 133 980, and EP 133 981; merocyanin derivatives such as those described in patent applications WO 04/006 878, WO 05/058 269, WO 06/032 741, FR 2 957 249, and FR 2 957 250; and mixtures thereof.

As examples of the (d) lipophilic organic UV filter, mention may be made of those denoted hereinbelow under their INCI names:

### Dibenzoylmethane Derivative:

Butylmethoxydibenzoylmethane or avobenzone sold under the trade name Parsol 1789 by the company DSM Nutritional Products,

### Para-Aminobenzoic Acid Derivatives:

Ethyl PABA,
Ethyl Dihydroxypropyl PABA,
Ethylhexyl Dimethyl PABA sold in particular under the name Escalol 507 by ISP,

### Salicylic Derivatives:

Homosalate sold under the name Eusolex HMS by Rona/EM Industries,
Ethylhexyl salicylate sold under the name Neo Heliopan OS by Symrise,

### Cinnamic Derivatives:

Ethylhexyl methoxycinnamate sold especially under the trade name Parsol MCX by DSM Nutritional Products,
Isopropyl methoxycinnamate,
Isoamyl methoxycinnamate sold under the trade name Neo Heliopan E 1000 by Symrise, Cinoxate,
Diisopropyl methylcinnamate,

### β,β-Diphenylacrylate Derivatives:

Octocrylene sold especially under the trade name Uvinul N539 by BASF, Etocrylene sold in particular under the trade name Uvinul N35 by BASF,

### Benzophenone Derivatives:

Benzophenone-1 sold under the trade name Uvinul 400 by BASF,
Benzophenone-2 sold under the trade name Uvinul D50 by BASF,
Benzophenone-3 or oxybenzone sold under the trade name Uvinul M40 by BASF,
Benzophenone-6 sold under the trade name Helisorb 11 by Norquay,
Benzophenone-8 sold under the trade name Spectra-Sorb UV-24 by American Cyanamid, Benzophenone-12,
N-hexyl 2-(4-diethylamino-2-hydroxybenzoyl)benzoate sold under the trade name Uvinul A+ or in the form of a mixture with octyl methoxycinnamate under the trade name Uvinul A+B by BASF,

### Benzylidenecamphor Derivatives:

3-Benzylidene Camphor manufactured under the name Mexoryl SD by Chimex,
4-Methylbenzylidene Camphor sold under the name Eusolex 6300 by Merck,
Polyacrylamidomethylbenzylidenecamphor manufactured under the name Mexoryl SW by Chimex,

### Phenylbenzotriazole Derivatives:

Drometrizole trisiloxane sold under the name Silatrizole by Rhodia Chimie,

### Triazine Derivatives:

Bis-(Ethylhexyloxyphenol) methoxyphenyl triazine sold under the trade name Tinosorb S by BASF,
Ethylhexyl triazone sold in particular under the trade name Uvinul T150 by BASF,
Diethylhexyl Butamido Triazone sold under the trade name Uvasorb HEB by Sigma 3V,
2,4,6-Tris(dineopentyl 4′-aminobenzalmalonate)-s-triazine,
2,4,6-Tris(diisobutyl 4′-aminobenzalmalonate)-s-triazine,
2,4-Bis(dineopentyl 4′-aminobenzalmalonate)-6-(n-butyl 4′-aminobenzoate)-s-triazine,
   the triazine silicones substituted by two aminobenzoate groups as those described in EP0841341 in particular 2,4-bis-(n-butyl 4′-aminobenzalmalonate)-6-[(3-{1,3,3,3-tetramethyl-1-[(trimethylsilyloxy]disiloxanyl}propyl)amino]-s-triazine,

### Anthranilic Derivatives:

Menthyl Anthranilate sold under the trade name Neo Heliopan MA by Symrise,

### Imidazoline Derivatives:

Ethylhexyl dimethoxybenzylidene dioxoimidazoline propionate,

### Benzalmalonate Derivatives:

Dineopentyl 4′-methoxybenzalmalonate,
Polyorganosiloxane containing benzalmalonate functions, for instance Polysilicone-15, sold under the trade name Parsol SLX by DSM,

### 4,4-Diarylbutadiene Derivatives:

1,1-dicarboxy(2,2′-dimethylpropyl)-4,4-diphenylbutadiene,

### Lipophilic Merocyanin Derivatives:

Octyl 5-N,N-diethylamino-2-phenylsulfonyl-2,4-pentadienoate and mixtures thereof.

Preferably, the (d) lipophilic organic UV filter may be chosen from:
Butylmethoxydibenzoylmethane,
Ethylhexyl methoxycinnamate,
Ethylhexyl salicylate,
Homosalate,
Butylmethoxydibenzoylmethane,
Octocrylene,
Benzophenone-3,
n-Hexyl 2-(4-diethylamino-2-hydroxybenzoyl)benzoate,
4-Methylbenzylidenecamphor,
Bis(ethylhexyloxyphenol)methoxyphenyltriazine,
Ethylhexyl triazone,
Diethylhexyl Butamido Triazone,
2,4-bis-(n-butyl 4′-aminobenzalmalonate)-6-[(3-{1,3,3,3-tetramethyl-1-[(trimethylsilyloxy]-disiloxanyl}propyl)amino]-s-triazine,
Drometrizole Trisiloxane,
Polysilicone-15,
1,1-dicarboxy(2,2′-dimethylpropyl)-4,4-diphenylbutadiene, and
mixtures thereof.

More preferably, the (d) lipophilic organic UV filter may be chosen from:
Butylmethoxydibenzoylmethane,
Ethylhexyl methoxycinnamate,
Octocrylene,
Ethylhexyl salicylate,
n-Hexyl 2-(4-diethylamino-2-hydroxybenzoyl)benzoate,
Bis(ethylhexyloxyphenol)methoxyphenyltriazine,
2,4-bis-(n-butyl 4′-aminobenzalmalonate)-6-[(3-{1,3,3,3-tetramethyl-1-[(trimethylsilyloxy]-disiloxanyl}propyl)amino]-s-triazine,
Ethylhexyl triazone,
Diethylhexyl Butamido Triazone,
Drometrizole trisiloxane, and
mixtures thereof.

In an embodiment, it may be preferable for the composition according to the present invention to include, as the (d) lipophilic organic UV filter, at least one selected from the group consisting of drometrizole trisiloxane, bis(ethylhexyloxyphenol)methoxyphenyltriazine, ethylhexyl triazone, diethylamino hydroxybenzoyl hexyl benzoate, butyl methoxybenzoylmethane, ethylhexyl methoxycinnamate, ethylhexyl salicylate, octocrylene, homosalate, and a mixture thereof.

The amount of the (d) lipophilic organic UV filter(s) in the composition according to the present invention may be 1% by weight or more, preferably 3% by weight or more, and more preferably 5% by weight or more, relative to the total weight of the composition.

On the other hand, the amount of the (d) lipophilic organic UV filter(s) in the composition according to the present invention may be 30% by weight or less, preferably 25% by weight or less, and more preferably 10% by weight or less, relative to the total weight of the composition.

The amount of the (d) lipophilic organic UV filter(s) in the composition according to the present invention may range from 1% to 30% by weight, preferably from 3% to 25% by weight, and more preferably from 5% to 20% by weight, relative to the total weight of the composition.

### [Oil]

The composition according to the present invention may comprise (e) at least one oil. If two or more oils are used, they may be the same or different.

The (e) oil is different from the (d) lipophilic organic UV filter.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (e) oil may be a non-polar oil such as a hydrocarbon oil, a silicone oil, or the like; a polar oil such as a plant or animal oil and an ester oil or an ether oil; or a mixture thereof.

The (e) oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously, and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used for the present invention are silicone oils as defined above, and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

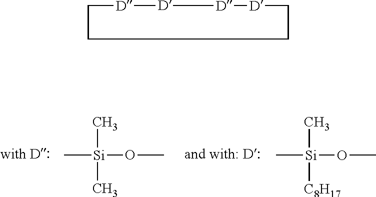

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

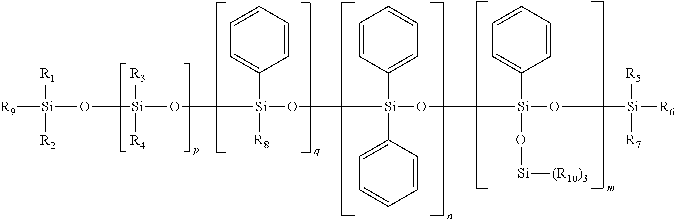

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers of 0 to 900 inclusive, preferably 0 to 500 inclusive, and more preferably 0 to 100 inclusive,
with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The hydrocarbon oils may be chosen from:
linear or branched, optionally cyclic, C₅-C₁₉ alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, and mixtures thereof.

It is also preferable that the (e) oil be chosen from oils with a molecular weight below 600 g/mol.

Preferably, the (e) oil has a low molecular weight such as below 600 g/mol, chosen among ester oils with a short hydrocarbon chain or chains (C₁-C₁₂) (e.g., isopropyl lauroyl sarcosinate, isopropyl myristate, isopropyl palmitate, isononyl isononanoate, and ethyl hexyl palmitate), silicone oils (e.g., volatile silicones such as cyclohexasiloxane), hydrocarbon oils (e.g., isododecane, isohexadecane, and squalane), branched and/or unsaturated fatty alcohol (C₁₂-C₃₀) type oils such as octyldodecanol and oleyl alcohol, and ether oils such as dicaprylyl ether.

The (e) oil may be chosen from polar oils, preferably from ester oils, and more preferably ethylhexyl palmitate, and preferably from artificial triglycerides, and more preferably caprylic/capric triglyceride.

The amount of the (e) oil(s) in the composition according to the present invention may be 1% by weight or more, preferably 5% by weight or more, and more preferably 10% by weight or more, relative to the total weight of the composition.

On the other hand, the amount of the (e) oil(s) in the composition according to the present invention may be 50% by weight or less, preferably 45% by weight or less, and more preferably 40% by weight or less, relative to the total weight of the composition.

The amount of the (e) oil(s) in the composition according to the present invention may range from 1% to 50% by weight, preferably from 5% to 45% by weight, more preferably from 10% to 40% by weight, relative to the total weight of the composition.

### [Nonionic Surfactant]

The composition according to the present invention may further comprise (f) at least one nonionic surfactant. A single type of nonionic surfactant may be used, but two or more different types of nonionic surfactant may be used in combination.

The (f) nonionic surfactant may further enhance the stability of the composition according to the present invention.

The (f) nonionic surfactant may be chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

The surfactant (1) may be a fluid at a temperature of less than or equal to 45° C.

The surfactant (1) may be selected in particular from:
polyglyceryl fatty acid esters of at least one, preferably one, fatty acid comprising at least one saturated or unsaturated, linear or branched C₈-C₂₂ hydrocarbon group such as C₈-C₂₂alkyl or alkenyl group, preferably C₈-C₁₈ alkyl or alkenyl group, and more preferably C₈-C₁₂ alkyl or alkenyl group, and of 2-12 glycerols, preferably 2-10 glycerols and more preferably 2-8 glycerols;
polyoxyethylenated (PEGylated) alkyl glycerides such as polyethylene glycol derivatives of a mixture of mono-, di- and tri-glycerides of caprylic and capric acids (preferably 2 to ethylene oxide units, more preferably 2 to 20 ethylene oxide units, and even more preferably 2 to 10 ethylene oxide units), e.g., PEG-6 Caprylic/Capric Glycerides, PEG-7 Caprylic/Capric Glycerides, and PEG-7 glyceryl cocoate;
polyoxyethylenated fatty ethers of at least one, preferably one, fatty alcohol comprising at least one saturated or unsaturated, linear or branched C₈-C₂₂ hydrocarbon group such as C₈-C₂₂alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and
mixtures thereof.

It may be preferable that the (f) nonionic surfactant be a mixture of at least two selected from the above polyglyceryl fatty acid esters, polyoxyethylenated alkyl glycerides, and polyoxyethylenated fatty ethers.

It is preferable that the polyglyceryl fatty acid ester have a polyglycerol moiety derived from 2 to 8 glycerols, more preferably from 2 to 6 glycerols, and even more preferably 2 to 4 glycerols.

The polyglyceryl fatty acid ester may be chosen from the mono, di and tri esters of saturated or unsaturated acid, preferably saturated acid, including 8 to 22 carbon atoms, preferably 8 to 18 carbon atoms, and more preferably 8 to 12 carbon atoms, such as caprylic acid, capric acid, lauric acid, oleic acid, stearic acid, isostearic acid, and myristic acid.

The polyglyceryl fatty acid ester may be selected from the group consisting of PG2 caprate, PG2 dicaprate, PG2 tricaprate, PG2 caprylate, PG2 dicaprylate, PG2 tricaprylate, PG2 laurate, PG2 dilaurate, PG2 trilaurate, PG2 myristate, PG2 dimyristate, PG2 trimyristate, PG2 stearate, PG2 distearate, PG2 tristearate, PG2 isostearate, PG2 diisostearate, PG2 triisostearate, PG2 oleate, PG2 dioleate, PG2 trioleare, PG3 caprate, PG3 dicaprate, PG3 tricaprate, PG3 caprylate, PG3 dicaprylate, PG3 tricaprylate, PG3 laurate, PG3 dilaurate, PG3 trilaurate, PG3 myristate, PG3 dimyristate, PG3 trimyristate, PG3 stearate, PG3 distearate, PG3 tristearate, PG3 isostearate, PG3 diisostearate, PG3 triisostearate, PG3 oleate, PG3 dioleate, PG3 trioleare, PG4 caprate, PG4 dicaprate, PG4 tricaprate, PG4 caprylate, PG4 dicaprylate, PG4 tricaprylate, PG4 laurate, PG4 dilaurate, PG4 trilaurate, PG4 myristate, PG4 dimyristate, PG4 trimyristate, PG4 stearate, PG4 distearate, PG4 tristearate, PG4 isostearate, PG4 diisostearate, PG4 triisostearate, PG4 oleate, PG4 dioleate, PG4 trioleare, PG5 caprate, PG5 dicaprate, PG5 tricaprate, PG5 caprylate, PG5 dicaprylate, PG5 tricaprylate, PG5 laurate, PG5 dilaurate, PG5 trilaurate, PG5 myristate, PG5 dimyristate, PG5 trimyristate, PG5 stearate, PG5 distearate, PG5 tristearate, PG5 isostearate, PG5 diisostearate, PG5 triisostearate, PG5 oleate, PG5 dioleate, PG5 trioleare, PG6 caprate, PG6 dicaprate, PG6 tricaprate, PG6 caprylate, PG6 dicaprylate, PG6 tricaprylate, PG6 laurate, PG6 dilaurate, PG6 trilaurate, PG6 myristate, PG6 dimyristate, PG6 trimyristate, PG6 stearate, PG6 distearate, PG6 tristearate, PG6 isostearate, PG6 diisostearate, PG6 triisostearate, PG6 oleate, PG6 dioleate, PG6 trioleare, PG10 caprate, PG10 dicaprate, PG10 tricaprate, PG10 caprylate, PG10 dicaprylate, PG10 tricaprylate, PG10 laurate, PG10 dilaurate, PG10 trilaurate, PG10 myristate, PG10 dimyristate, PG10 trimyristate, PG10 stearate, PG10 distearate, PG10 tristearate, PG10 isostearate, PG10 diisostearate, PG10 triisostearate, PG10 oleate, PG10 dioleate, and PG10 trioleare.

The polyoxyalkylenated fatty ethers, preferably polyoxyethylenated fatty ethers, may comprise from 2 to 60 ethylene oxide units, preferably from 2 to 30 ethylene oxide units, and more preferably from 2 to 10 ethylene oxide units. The fatty chain of the ethers may be chosen in particular from lauryl, behenyl, arachidyl, stearyl and cetyl units, and mixtures thereof, such as cetearyl. Examples of ethoxylated fatty ethers which may be mentioned are lauryl alcohol ethers comprising 2, 3, 4, and 5 ethylene oxide units (CTFA names: Laureth-2, Laureth-3, Laureth-4, and Laureth-5, such as the products sold under the names Nikkol BL-2 by the company Nikko Chemicals, Emalex 703 by the company Nihon Emulsion Co., Ltd, Nikkol BL-4 by the company Nildo Chemicals, and EMALEX 705 by the company Nihon Emulsion Co., Ltd.

The (2) mixed esters of fatty acids, or of fatty alcohol, of carboxylic acid and of glycerol, which can be used as the above nonionic surfactant, may be chosen in particular from the group comprising mixed esters of fatty acid or of fatty alcohol with an alkyl or alkenyl chain containing from 8 to 22 carbon atoms, preferably from 8 to 18 carbon atoms, and more preferably from 8 to 12 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the nanoemulsion of the invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the nanoemulsion of the invention, mention may be made of the mixed ester of glycerol and of the mixture of citric acid, lactic acid, linoleic acid and oleic acid (CTFA name: Glyceryl citrate/lactate/linoleate/oleate) sold by the company Hüls under the name Imwitor 375; the mixed ester of succinic acid and of isostearyl alcohol with glycerol (CTFA name: Isostearyl diglyceryl succinate) sold by the company Hols under the name Imwitor 780 K; the mixed ester of citric acid and of stearic acid with glycerol (CTFA name: Glyceryl stearate citrate) sold by the company Hills under the name Imwitor 370; the mixed ester of lactic acid and of stearic acid with glycerol (CTFA name: Glyceryl stearate lactate) sold by the company Danisco under the name Lactodan B30 or Rylo LA30.

The (3) fatty acid esters of sugars, which can be used as the above nonionic surfactant, may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the present invention comprise a saturated or unsaturated linear alkyl or alkenyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, Fl10 and F160; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The (3) fatty alcohol ethers of sugars, which can be used as the above nonionic surfactant, may be solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the nanoemulsion of the invention comprise a saturated or unsaturated, linear alkyl or alkenyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

The (4) fatty esters of sorbitan and oxyalkylenated fatty esters of sorbitan which may be used as the above nonionic surfactant may be chosen from the group comprising C₁₆-C₂₂ fatty acid esters of sorbitan and oxyethylenated C₁₆-C₂₂ fatty acid esters of sorbitan. They may be formed from at least one fatty acid comprising at least one saturated linear alkyl chain containing, respectively, from 16 to 22 carbon atoms, and from sorbitol or from ethoxylated sorbitol. The oxyethylenated esters may generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of the above nonionic surfactant can be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The (4) oxyalkylenated fatty esters, preferably ethoxylated fatty esters, which may be used as the above nonionic surfactant, may be esters formed from 1 to 100 ethylene oxide units, preferably from 2 to 60 ethylene oxide units, and more preferably from 2 to 30 ethyle oxide units, and from at least one fatty acid chain containing from 8 to 22 carbon atoms, preferably from 8 to 18 carbon atoms, and more preferably from 8 to 12 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The (5) block copolymers of ethylene oxide (A) and of propylene oxide (B), which may be used as the above nonionic surfactant, may be chosen in particular from block copolymers of formula (I):

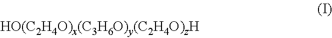

in which x, y and z are integers such that x+z ranges from 2 to 100 and y ranges from 14 to 60, and mixtures thereof, and more particularly from the block copolymers of formula (I) having an HLB value ranging from 8.0 to 14.0.

The (6) polyoxyethylenated (1-40 EO) and polyoxypropylenated (1-30 PO) alkyl (C₁₆-C₃₀) ethers, which may be used as the above nonionic surfactant, may be selected from the group consisting of:
PPG-6 Decyltetradeceth-30; Polyoxyethlene (30) Polyoxypropylene (6) Tetradecyl Ether such as those sold as Nikkol PEN-4630 from Nikko Chemicals Co.,
PPG-6 Decyltetradeceth-12; Polyoxyethylene (12) Polyoxypropylene (6) Tetradecyl Ether such as those sold as Nikkol PEN-4612 from Nikko Chemicals Co.,
PPG-13 Decyltetradeceth-24; Polyoxyethylene (24) Polyoxypropylene (13) Decyltetradecyl Ether such as those sold as UNILUBE 50MT-2200B from NOF Corporation,
PPG-6 Decyltetradeceth-20; Polyoxyethylene (20) Polyoxypropylene (6) Decyltetradecyl Ether such as those sold as Nildcol PEN-4620 from Nikko Chemicals Co.,
PPG-4 Ceteth-1; Polyoxyethylene (1) Polyoxypropylene (4) Cetyl Ether such as those sold as Nikkol PBC-31 from Nikko Chemicals Co.,
PPG-8 Ceteth-1; Polyoxyethylene (1) Polyoxypropylene (8) Cetyl Ether such as those sold as Nildcol PBC-41 from Nikko Chemicals Co.,
PPG-4 Ceteth-10; Polyoxyethylene (10) Polyoxypropylene (4) Cetyl Ether such as those sold as Nikkol PBC-33 from Nikko Chemicals Co.,
PPG-4 Ceteth-20; Polyoxyethylene (20) Polyoxypropylene (4) Cetyl Ether such as those sold as Nikkol PBC-34 from Nikko Chemicals Co.,
PPG-5 Ceteth-20; Polyoxyethylene (20) Polyoxypropylene (5) Cetyl Ether such as those sold as Procetyl AWS from Croda Inc.,
PPG-8 Ceteth-20; Polyoxyethylene (20) Polyoxypropylene (8) Cetyl Ether such as those sold as Nikkol PBC-44 from Nikko Chemicals Co., and
PPG-23 Steareth-34; Polyoxyethylene Polyoxypropylene Stearyl Ether (34 EO) (23 PO) such as those sold as Unisafe 34S-23 from Pola Chemical Industries. They can provide a composition with stability for a long time, even though the temperature of the composition is increased and decreased in a relatively short period of time.

It may be more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It may be even more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20.

As (7) silicone surfactants, which may be used as the above nonionic surfactant, mention may be made of those disclosed in documents U.S. Pat. Nos. 5,364,633 and 5,411,744.

The (7) silicone surfactant as the above nonionic surfactant may preferably be a compound of formula (I):

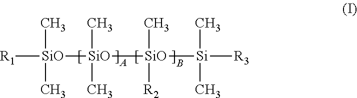

in which:
R₁, R₂ and R₃, independently of each other, represent a C₁-C₆ alkyl radical or a radical —(CH₂)ₓ—(OCH₂CH₂)_{y}—(OCH₂CH₂CH₂)—OR₄, at least one radical R¹, R² or R₃ not being an alkyl radical; R⁴ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30;
z is an integer ranging from 0 to 5.

According to one preferred embodiment of the invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

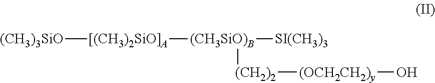

in which A is an integer ranging from 20 to 105, B is an integer ranging from 2 to 10 and y is an integer ranging from 10 to 20.

As examples of silicone surfactants of formula (I), mention may also be made of the compounds of formula (III):

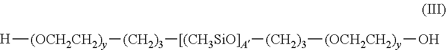

in which A′ and y are integers ranging from 10 to 20.

Compounds of the invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

The (f) nonionic surfactant may have an HLB (Hydrophilic Lipophilic Balance) value of from 8.0 to 14.0, preferably from 9.0 to 13.5, and more preferably from 10.0 to 13.0. If two or more nonionic surfactants are used, the HLB value is determined by the weighted average of the HLB values of all the nonionic surfactants.

It is preferable that the (f) nonionic surfactant be selected from polyglyceryl fatty acid esters, and more preferably polyglyceryl fatty acid monoesters.

In particular, the (f) nonionic surfactant may be a mixture of a first polyglyceryl fatty acid monoester with an HLB value of less than 10, preferably less than 9, and more preferably less than 8, and a second polyglyceryl fatty acid monoester with an HLB value of more than 11, preferably more than 12, and more preferably more than 13.

The amount of the (f) nonionic surfactant(s) in the composition according to the present invention may be 0.01% by weight or more, preferably 0.05% by weight or more, and more preferably 0.1% by weight or more relative to the total weight of the composition.

On the other hand, the amount of the (f) nonionic surfactant(s) in the composition according to the present invention may be 5% by weight or less, preferably 3% by weight or less, and more preferably 1% by weight or less relative to the total weight of the composition.

The amount of the (f) nonionic surfactant(s) in the composition according to the present invention may range from 0.01% to 5% by weight, preferably from 0.05% to 3% by weight, more preferably from 0.1% to 1% by weight relative to the total weight of the composition.

### [Inorganic Particle]

The composition according to the present invention may further comprise (g) at least one inorganic particle. A single type of inorganic particle may be used, but two or more different types of inorganic particle may be used in combination.

The particle size of the (g) inorganic particle corresponds to a primary particle size. In addition, the particle size here means an average or mean particle size, which may be a volume-average particle size, which can be determined by, for example, a laser diffraction particle size analyzer.

The particle size of the (g) inorganic particle used for the present invention is not limited. However, it is preferable that the particle size of the (f) inorganic particle be 50 μm or less, preferably 30 μm or less, more preferably 15 μm or less, even more preferably 10 μm or less, and particularly preferably from 2 to 5 μm.

The (g) inorganic particle may or may not be porous, preferably non-porous.

The (g) inorganic particle may be hollow or solid, preferably hollow.

The (g) inorganic particle may comprise or consists of at least one material selected from:
metal oxides such as zirconium oxides, cerium oxides, iron oxides and titanium oxides,
alumina,
silicates such as talc, clays and kaolin,
glass particles,
silica (silicon dioxide),
calcium carbonate or magnesium carbonate,
magnesium hydrogen carbonates,
hydroxyapatite, and
mixtures thereof.

Among the silica particles, mention may be made of hollow spherical silica particles such as the products sold under the trade name BA4 from the company JGC Catalysts and Chemicals Ltd. in Japan.

The amount of the (g) inorganic particle(s) in the composition according to the present invention may be 1% by weight or more, preferably 2% by weight or more, and more preferably 3% by weight or more, relative to the total weight of the composition.

On the other hand, the amount of the (g) inorganic particle(s) in the composition according to the present invention may be 20% by weight or less, preferably 15% by weight or less, and more preferably 10% by weight or less, relative to the total weight of the composition.

The amount of the (g) inorganic particle(s) in the composition according to the present invention may range from 1% to 20% by weight, preferably from 2% to 15% by weight, more preferably from 3% to 10% by weight, relative to the total weight of the composition.

### [Additional Optional Ingredients]

The composition according to the present invention may also comprise an effective amount of additional optional ingredient(s), known previously elsewhere in cosmetic compositions, for example, anionic, cationic or amphoteric surfactants; organic powders different from the ingredient (a); thickeners different from the ingredient (b); sequestering agents such as EDTA and trisodium ethylenediamine disuccinate; preserving agents; vitamins or provitamins, for instance, panthenol; fragrances; plant extracts; and so on.

The composition according to the present invention may include one or several cosmetically acceptable organic solvents, which may be alcohols: in particular monovalent alcohols such as ethyl alcohol, isopropyl alcohol, benzyl alcohol, phenoxyethanol and phenylethyl alcohol; diols such as ethylene glycol, caprylyl glycol, propylene glycol, dipropylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The organic water-soluble solvents may be present in an amount ranging from 0.01% by weight or more, preferably 0.1% by weight or more, and more preferably 1% by weight or more, relative to the total weight of the composition.

The organic water-soluble solvents may be present in an amount ranging from 20% by weight or less, preferably 15% by weight or less, and more preferably 10% by weight or less, relative to the total weight of the composition.

The organic water-soluble solvents may be present in an amount ranging from 0.01% to 20% by weight, preferably from 0.1% to 15% by weight, and more preferably from 1% to 10% by weight, relative to the total weight of the composition.

### [Preparation and Properties]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention. The conventional method and means may include a homogenizer, for example a turbine mixer.

### [Form]

The composition according to the present invention may be in various forms.

As the composition according to the present invention includes (c) water, the composition according to the present invention may be, for example, a fluid such as an aqueous solution. The aspect of the composition according to the present invention may be a gel, because the (b) associative polymer can function as a thickener.

The composition according to the present invention may be in the form of an O/W emulsion, if it includes (d) at least one lipophilic organic UV filter and/or (e) at least one oil. The aspect of the composition according to the present invention in the form of an O/W emulsion that may be a paste or a cream.

The composition according to the present invention in the form of an O/W emulsion comprises dispersed fatty phases such as oil phases dispersed in a continuous aqueous phase. The dispersed fatty phases can be in the form of oil droplets in the aqueous phase.

The aqueous phase may be thickened, because the (b) associative polymer can function as a thickener. It is preferable that the composition according to the present invention be in the form of an O/W gel emulsion.

The O/W architecture or structure, which consists of fatty phases dispersed in an aqueous phase, has an external aqueous phase, and therefore, the composition according to the present invention with the O/W architecture or structure can provide a pleasant feeling during use because of the feeling of immediate freshness that the aqueous phase can provide.

It is preferable that the composition according to the present invention have a viscosity of 10,000 mPa·s or more, more preferably 15,000 mPa·s or more, and even more preferably 20,000 mPa·s or more. The viscosity can be measured with a B-type viscometer (for example, Rotor: No. 4, Rotation speed: 6 rpm, Measurement time: 1 minute) at 25° C.

It is also preferable that the composition according to the present invention have a viscosity at 25° C. of 200,000 mPa·s or less, more preferably 150,000 mPa·s or less, and even more preferably 100,000 mPa·s or less.

It may be preferable that the composition according to the present invention have a viscosity at 25° C. of from 10,000 mPa·s to 200,000 mPa·s, more preferably from 15,000 mPa·s to 150,000 mPa·s, and even more preferably from 20,000 mPa·s to 100,000 mPa·s.

### [Process and Use]

It is preferable that the composition according to the present invention be a cosmetic composition, and more preferably a cosmetic composition for a keratin substance such as skin.

The composition according to the present invention can be used for a non-therapeutic process, such as a cosmetic process, for treating a keratin substance such as skin, hair, mucous membranes, nails, eyelashes, eyebrows and/or scalp, by being applied to the keratin substance.

Thus, the present invention also relates to a cosmetic process for treating a keratin substance, preferably skin, comprising the step of applying the composition according to the present invention to the keratin substance.

The present invention may also relate to a use of the composition according to the present invention as a cosmetic product or in a cosmetic product such as care products, for body and/or facial skin and/or mucous membranes and/or the scalp and/or the hair and/or the nails and/or the eyelashes and/or the eyebrows.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The care product may be a lotion, a cream, and the like.

The present invention may also relate to a use of (a) at least one cellulose particle in a composition comprising:
(b) at least one associative polymer comprising one or more acrylic and/or methacrylic units; and
(c) water,
in order to cause texture transformation of the composition when being applied onto a keratin substance such as skin, preferably at the early stage of the application, and/or reduce the stickiness of the keratin substance after application.

The present invention may also relate to a use of (b) at least one associative polymer comprising one or more acrylic and/or methacrylic units,
in a composition comprising:
(a) at least one cellulose particle; and
(c) water,
in order to cause texture transformation of the composition when being applied onto a keratin substance such as skin, preferably at the early stage of the application, and/or stabilize the composition.

The explanations regarding the ingredients (a) to (c) in the composition according to the present invention can apply to those in the above use according to the present invention.

The present invention may also relate to a use of (a) at least one cellulose particle in a composition comprising:
(b) at least one associative polymer comprising one or more acrylic and/or methacrylic units;
(c) water; and
(d) at least one lipophilic organic UV filter and/or (e) at least one oil,
in order to cause texture transformation of the composition when being applied onto a keratin substance such as skin, preferably at the early stage of the application, and/or stabilize the composition, and/or reduce the stickiness, oiliness/greasiness, and shiny aspect of the keratin substance after application.

The present invention may also relate to a use of (b) at least one associative polymer comprising one or more acrylic and/or methacrylic units,
in a composition comprising:
(a) at least one cellulose particle;
(c) water; and
(d) at least one lipophilic organic UV filter and/or (e) at least one oil,
in order to cause texture transformation of the composition when being applied onto a keratin substance such as skin, preferably at the early stage of the application, and/or stabilize the composition.

The explanations regarding the ingredients (a) to (e) in the composition according to the present invention can apply to those in the above use according to the present invention.

It may be preferable that the composition used in the above use according to the present invention include (f) at least one nonionic surfactant.

It may be preferable that the composition used in the above use according to the present invention include (g) at least one inorganic particle.

### EXAMPLES

The present invention will be described in more detail by way of examples, which however should not be construed as limiting the scope of the present invention.

### Example 1A and Comparative Examples 1A-6A

The following compositions, in the form of an aqueous gel, according to Example 1A and Comparative Examples 1A-6A shown in Table 1A, were prepared by mixing the components shown in Table 1A as follows:
(1) mixing the ingredients in the rows of A in Table 1A at 75-80° C. to form a uniform mixture of Phase A;
(2) adding the ingredient in the rows of B in Table 1A to the mixture of Phase A, followed by homogenizing them to obtain a uniform mixture (Phases A and B);
(3) adding the ingredient in the rows of C in Table 1A to the mixture of Phases A and B, followed by homogenizing them to obtain a uniform mixture (Phases A, B and C) and cooling the obtained mixture to a room temperature (25° C.); and
(4) adding the ingredient in the row of D in Table 1A to the mixture of Phases A, B and C, followed by homogenizing them to obtain a uniform mixture (Phases A, B, C and D).

The numerical values for the amounts of the components shown in Table 1A are all based on “% by weight” as raw materials.

TABLE 1A

| | | | | Comp. | Comp. | Comp. | Comp. | Comp. | Comp. |
|---|---|---|---|---|---|---|---|---|---|
| Phase | | Ingredient | Ex. 1A | Ex. 1A | Ex. 2A | Ex. 3A | Ex. 4A | Ex. 5A | Ex. 6A |
| A | | Glycerin | 5 | 5 | 5 | 5 | 5 | 5 | 5 |
| | | Dipropylene Glycol | 5 | 5 | 5 | 5 | 5 | 5 | 5 |
| | | Caprylyl Glycol | 0.2 | 0.2 | 0.2 | 0.2 | 0.2 | 0.2 | 0.2 |
| | | Phenoxyethanol | 0.7 | 0.7 | 0.7 | 0.7 | 0.7 | 0.7 | 0.7 |
| | | Trisodium Ethylenediamine Disuccinate | 0.1 | 0.1 | 0.1 | 0.1 | 0.1 | 0.1 | 0.1 |
| | (c) | Water | qsp 100 | qsp 100 | qsp 100 | qsp 100 | qsp 100 | qsp 100 | qsp 100 |
| B | (a) | Cellulose | 5 | — | — | — | — | 5 | 5 |
| | | Silica | | | | | 5 | — | — |
| C | (b) | Acrylates/Beheneth-25 Methacrylate Copolymer | 1.67 | 1.67 | 1.76 | 2.5 | 1.67 | — | — |
| | | (30 wt % AM in water (70 wt %)) | | | | | | | |
| | | Ammonium Polyacryloyldimethyl Taurate | — | — | — | — | — | — | 0.5 |
| D | | Sodium Hydroxide | qs pH 7 | qs pH 7 | qs pH 7 | qs pH 7 | qs pH 7 | qs pH 7 | qs pH 7 |

| | | | | | | | |
|---|---|---|---|---|---|---|---|
| Viscosity (mPa · s) | 22495 | 6399 | 10998 | 57188 | 8998 | 4 | 10298 |
| Texture Transformation | Very | Very | Poor | Poor | Poor | Very | Poor |
| | Good | Poor | | | | Poor | |
| Non-Sticky Feeling | Very | Poor | Poor | Very | Very | Very | Very |
| | Good | | | Poor | Good | Good | Good |
| Stability | Very | Very | Very | Very | Very | Very | Very |
| | Good | Good | Good | Good | Good | Poor | Good |

AM: Active Material

The properties of the ingredient (a) (cellulose particles) in Table 1A are shown in Table 2A.

TABLE 2A

| | | Mean Size | WP Oil | WP Water |
|---|---|---|---|---|
| Powder | Shape | (μm) | (ml/100 g) | (ml/100 g) |
| Cellulose | Sphere | 4.0 | 296.0 | 400.8 |
| (Cellulobeads USF, | | | | |
| Daito Kasei) | | | | |

Mean Size: Number-Average Primary Particle Size
WP Oil: Wet Point for Oil
WP Water: Wet Point for Water

### (Wet Point for Oil)

The wet point for oil was determined by the following protocol.
(1) 2 g of the powder component was kneaded with a spatula on a glass plate while adding isononyl isononanoate with a viscosity of 9 cP at 25° C. and a density of 0.853 g/ml.
(2) When the powder component became completely wet and started to form a paste, the weight of the added oil was determined as the weight of the wet point.
(3) The wet point for oil was calculated from the equation: The wet point for oil (ml/100 g)={(the weight of wet point)/2 g}×100/the density of oil.

### (Wet Point for Water)

The wet point for water was determined by the following protocol.
(1) 2 g of the powder component was kneaded with a spatula on a glass plate while adding water with a density of 0.998 g/ml.
(2) When the powder component became completely wet and started to form a paste, the weight of the added water was determined as the weight of the wet point.
(3) The wet point for water was calculated from the equation: The wet point for water (ml/100 g)={(the weight of the wet point)/2 g}×100/the density of water.

### [Evaluations]

### (Viscosity)

The viscosity of each of the compositions according to Example 1A and Comparative Examples 1A-6A was measured at a room temperature (25° C.) with a B-type viscometer under the conditions of rotor: No. 4, rotation speed: 6 rpm, and measurement time: 1 minute.

The results are shown in Table 1A.

### (Texture Transformation)

Five professional panelists evaluated “texture transformation” during the application of the compositions according to Example 1A and Comparative Examples 1A-6A. Each panelist took each composition and applied it circularly onto their faces, They evaluated the timing when texture transformation (from gel to liquid) occurred, and graded it from 1 (poor) to 5 (very good), which was then classified in the following 4 categories based on the average of the grade:
Very Good: From 5.0 to 4.0 (texture transformation is clearly felt at the early stage of application)
Good: From 3.9 to 3.0 (texture transformation is felt at the early stage of application)
Poor: From 2.9 to 2.0 (texture transformation is hardly felt at the early stage of application)
Very Poor: From 1.9 to 1.0 (texture transformation is not felt at all at the early stage of application)

The results are shown in Table 1A.

### (Non-Sticky Feeling)

Five professional panelists evaluated the “non-sticky feeling” after application of the compositions according to Example 1A and Comparative Examples 1A-6A, Each panelist took each composition and applied it onto their faces to evaluate the non-sticky feeling after application, and graded it from 1 (poor) to 5 (very good), which was then classified in the following 4 categories based on the average of the grade:
Very Good: From 5.0 to 4.0 (stickiness is not felt at all after application)
Good: From 3.9 to 3.0 (stickiness is hardly felt after application)
Poor: From 2.9 to 2.0 (stickiness is felt after application)
Very Poor: From 1.9 to 1.0 (stickiness is clearly felt after application)

The results are shown in Table 1A.

### (Stability)

Each of the compositions according to Example 1A and Comparative Examples 1A-6A was filled into a glass bottle and was held under temperature changing conditions at 4° C., 25° C., 37° C. and 45° C. for 2 months. Each sample was then investigated for the degree of change (aspect, color, and odor), and evaluated in accordance with the following criteria:
Very Good: Almost the same conditions as at production.
Good: Little changes in aspect, color, and odor were observed.
Poor: Changes in aspect, color, and odor were clearly observed.
Very Poor: Changes in aspect, color, and odor were remarkably noticed.

The results are shown in Table 1A.

### SUMMARY

As is clear from Table 1A, the composition in the form of an aqueous gel according to the present invention (Example 1A) was able to provide excellent cosmetic effects in terms of texture transformation, non-sticky feeling, and stability under temperature changes for 2 months, which could be attributed to a combination of the ingredients (a) and (b) under the presence of the ingredient (c). Thus, the composition according to the present invention can provide, in particular, an excellent feeling to the touch and stability for a long time period even under temperature changes.

On the other hand, the compositions according to Comparative Examples 1A-3A which did not include the ingredient (a) (cellulose particles) showed inferior texture transformation and stickiness.

The composition according to Comparative Example 4A which included silica particles instead of the ingredient (a) showed inferior texture transformation.

The composition according to Comparative Example 5A which did not include the ingredient (b) (acrylates/beheneth-25 methacrylate copolymer) could not be thickened, and showed inferior texture transformation and instability.

The composition according to Comparative Example 6A which included another thickener instead of the ingredient (b) showed inferior texture transformation.

### Example 2A and Comparative Examples 7A-10A

The following compositions, in the form of a cream (an O/W emulsion), according to Example 2A and Comparative Examples 7A-10A, shown in Table 3A, were prepared by mixing the components shown in Table 3A as follows:
(1) mixing the ingredients in the rows of A in Table 3A at 75-80° C. to form a uniform mixture of Phase A;
(2) adding the ingredient in the rows of B in Table 3A to the mixture of Phase A, followed by homogenizing them to obtain a uniform mixture (Phases A and B);
(3) adding the ingredient in the rows of C in Table 3A to the mixture of Phases A and B, followed by homogenizing them to obtain a uniform mixture (Phases A, B and C) and cooling the obtained mixture to a room temperature (25° C.);
(4) adding the ingredients in the rows of E in Table 3A to the mixture of Phases A, B, and C, followed by homogenizing them to obtain a uniform mixture (Phases A, B, C and E); and
(5) adding the ingredient in the row of D in Table 3A to the mixture of Phases A, B, C and E, followed by homogenizing them to obtain a uniform mixture (Phases A, B, C, D and E).

The numerical values for the amounts of the components shown in Table 3A are all based on “% by weight” as raw materials.

TABLE 3A

| | | | | Comp. | Comp. | Comp. | Comp. |
|---|---|---|---|---|---|---|---|
| Phase | | Ingredient | Ex. 2A | Ex. 7A | Ex. 8A | Ex. 9A | Ex. 10A |
| A | | Glycerin | 3 | 3 | 3 | 3 | 3 |
| | | Dipropylene Glycol | 3 | 3 | 3 | 3 | 3 |
| | | Caprylyl Glycol | 0.2 | 0.2 | 0.2 | 0.2 | 0.2 |
| | | Phenoxyethanol | 0.7 | 0.7 | 0.7 | 0.7 | 0.7 |
| | | Trisodium Ethylenediamine Disuccinate | 0.1 | 0.1 | 0.1 | 0.1 | 0.1 |
| | (c) | Water | qsp 100 | qsp 100 | qsp 100 | qsp 100 | qsp 100 |
| B | (a) | Cellulose | 2.75 | — | — | 2.75 | 2.75 |
| | | Silica | — | — | 2.75 | — | — |
| C | (b) | Acrylates/Beheneth-25 Methacrylate Copolymer | 1 | 1 | 1 | — | — |
| | | (30 wt % AM in water (70wt %)) | | | | | |
| | | Ammonium Polyacryloyldimethyl Taurate | — | — | — | — | 0.3 |
| D | | Sodium Hydroxide | qs pH 7 | qs pH 7 | qs pH 7 | qs pH 7 | qs pH 7 |
| E | (e) | Caprylic/Capric Triglyceride | 20 | 20 | 20 | 20 | 20 |
| | (e) | Ethylhexyl Palmitate | 20 | 20 | 20 | 20 | 20 |

| | | | | | |
|---|---|---|---|---|---|
| Viscosity (mPa · s) | 16496 | 2699 | 6928 | NA | 7652 |
| Texture Transformation | Good | Very | Poor | Very | Very |
| | | Poor | | Poor | Poor |
| Non-Sticky Feeling | Very | Poor | Very | Very | Very |
| | Good | | Good | Good | Good |
| Stability | Very | Poor | Poor | Very | Poor |
| | Good | | | Poor | |

AM: Active Material,
NA: Not Available

The properties of the cellulose (particles) in Table 3A are the same as those shown in Table 2A.

### [Evaluations]

### (Viscosity)

The viscosity of each of the compositions according to Example 2A and Comparative Examples 7A, 8A and 10A was measured at a room temperature (25° C.) with a B-type viscometer under the conditions of rotor: No. 4, rotation speed: 6 rpm, and measurement time: 1 minute.

The viscosity of the composition according to Comparative Example 9A could not be measured due to a phase separation of the composition.

The results are shown in Table 3A.

### (Texture Transformation)

Five professional panelists evaluated “texture transformation” during the application of the compositions according to Example 2A and Comparative Examples 7A-10A. Each panelist took each composition and applied it circularly onto their faces. They evaluated the timing when texture transformation (from cream to liquid) occurred, and graded it from 1 (poor) to 5 (very good), which was then classified in the following 4 categories based on the average of the grade:
Very Good: From 5.0 to 4.0 (texture transformation is clearly felt at the early stage of application)
Good: From 3.9 to 3.0 (texture transformation is felt at the early stage of application)
Poor: From 2.9 to 2.0 (texture transformation is hardly felt at the early stage of application)
Very Poor: From 1.9 to 1.0 (texture transformation is not felt at all at the early stage of application)

The results are shown in Table 3A.

### (Non-Sticky Feeling)

Five professional panelists evaluated the “non-sticky feeling” after application of the compositions according to Example 2A and Comparative Examples 7A-10A. Each panelist took each composition and applied it onto their faces to evaluate the non-sticky feeling after application, and graded it from 1 (poor) to 5 (very good), which was then classified in the following 4 categories based on the average of the grade:
Very Good: From 5.0 to 4.0 (stickiness is not felt at all after application)
Good: From 3.9 to 3.0 (stickiness is hardly felt after application)
Poor: From 2.9 to 2.0 (stickiness is felt after application)
Very Poor: From 1.9 to 1.0 (stickiness is clearly felt after application)

The results are shown in Table 3A.

### (Stability)

Each of the compositions according to Example 2A and Comparative Examples 7A-10A was filled into a glass bottle and was held under temperature changing conditions at 4° C., 25° C., 37° C. and 45° C. for 2 months. Each sample was then investigated for the degree of change (aspect, color, and odor), and evaluated in accordance with the following criteria:
Very Good: Almost the same conditions as at production.
Good: Little changes in aspect, color, and odor were observed.
Poor: Changes in aspect, color, and odor were clearly observed,
Very Poor: Changes in aspect, color, and odor were remarkably noticed.

The results are shown in Table 3A.

### Summary

As is clear from Table 3A, the composition in the form of a cream according to the present invention (Example 2A) was able to provide excellent cosmetic effects in terms of texture transformation, non-sticky feeling, and stability under temperature changes for 2 months, which could be attributed to a combination of the ingredients (a) and (b) under the presence of the ingredient (c). Thus, the composition according to the present invention can provide, in particular, an excellent feeling to the touch and stability for a long time period even under temperature changes.

On the other hand, the composition according to Comparative Example 7A which did not include the ingredient (a) (cellulose particles) showed inferior texture transformation and stickiness.

The composition according to Comparative Example 8A which included silica particles instead of the ingredient (a) showed inferior texture transformation and instability.

The composition according to Comparative Example 9A which did not include the ingredient (b) (acrylates/beheneth-25 methacrylate copolymer) showed inferior texture transformation and instability.

The composition according to Comparative Example 10A which included another thickener instead of the ingredient (b) showed inferior texture transformation and instability.

### Example 1B-2B and Comparative Examples 1B-2B

The following compositions, in the form of an O/W emulsion, according to Examples 1B-2B and Comparative Examples 1B-2B shown in Table 1B, were prepared by mixing the components shown in Table 1B as follows:
(1) mixing the ingredients in the rows of A in Table 1B at 75-80° C. to form a uniform mixture of Phase A;
(2) adding the ingredient in the rows of B in Table 1B to the mixture of Phase A, followed by homogenizing them to obtain a uniform mixture (Phases A and B);
(3) adding the ingredient, if present, in the rows of C in Table 1B to the mixture of Phases A and B, followed by homogenizing them to obtain a uniform mixture (Phases A, B and C);
(4) adding the ingredients in the rows of D in Table 1B to the mixture of Phases A and B, followed by homogenizing them to obtain a uniform mixture (Phases A, B, C and D), and cooling the obtained mixture to a room temperature (25° C.);
(5) adding the ingredients in the row of E in Table 1B to the mixture of Phases A, B, C and D, followed by homogenizing them to obtain a uniform mixture (Phases A, B, C, D and E);
(6) adding the ingredient in the row of F in Table 1B to the mixture of Phases A, B, C, D and E, followed by homogenizing them to obtain a uniform mixture (Phases A, B, C, D, E and F); and
(7) adding the ingredient in the row of G in Table 1B to the mixture of Phases A, B, C, D, E and F, followed by homogenizing them to obtain a uniform mixture (Phases A, B, C, D, E, F and G) with a pH of 7.0.

The numerical values for the amounts of the components shown in Table 1B are all based on “% by weight” as raw materials.

TABLE 1B

| | | | | | Comp. | Comp. |
|---|---|---|---|---|---|---|
| Phase | | Ingredient | Ex. 1B | Ex. 2B | Ex. 1B | Ex. 2B |
| A | | Glycerin | 5 | 5 | 5 | 5 |
| | | Propanediol | 2.5 | 2.5 | 2.5 | 2.5 |
| | | Caprylyl Glycol | 0.3 | 0.3 | 0.3 | 0.3 |
| | | Phenoxyethanol | 0.6 | 0.6 | 0.6 | 0.6 |
| | | Hydroxyacetophenone | 0.5 | 0.5 | 0.5 | 0.5 |
| | | Trisodium Ethylenediamine Disuccinate | 0.1 | 0.1 | 0.1 | 0.1 |
| | (c) | Water | qsp 100 | qsp 100 | qsp 100 | qsp 100 |
| | (f) | Polyglyceryl-2 Oleate | 0.24 | 0.24 | 0.24 | 0.24 |
| | (f) | Polyglyceryl-4 Caprate | 0.57 | 0.57 | 0.57 | 0.57 |
| B | (a) | Cellulose | 2.75 | 2.75 | — | 2.75 |
| C | (g) | Silica | 4 | — | 4 | 4 |
| D | (d) | Bis-Ethylhexyloxyphenol Methoxyphenyl Triazine | 3 | 3 | 3 | 3 |
| | (d) | Ethylhexyl Triazone | 4.5 | 4.5 | 4.5 | 4.5 |
| | (d) | Butyl Methoxydibenzoylmethane | 2 | 2 | 2 | 2 |
| | (d) | Drometrizole Trisiloxane | 5 | 5 | 5 | 5 |
| | (d) | Diethylamino Hydroxybenzoyl Hexyl Benzoate | 1 | 1 | 1 | 1 |
| | (e) | Isopropyl Myristate | 5 | 5 | 5 | 5 |
| | (e) | Diisopropyl Sebacate | 5 | 5 | 5 | 5 |
| | (e) | C15-19 Alkane | 3 | 3 | 3 | 3 |
| | (e) | Dicaprylyl Carbonate | 8 | 8 | 8 | 8 |
| | (e) | Isopropyl Lauroyl Sarcosinate | 4 | 4 | 4 | 4 |
| E | (b) | Acrylates/Beheneth-25 Methacrylate Copolymer | 0.8 | 0.8 | 0.8 | — |
| | | (30 wt % AM in water (70 wt %)) | | | | |
| | | Ammonium Polyacryloyldimethyl Taurate | 0.15 | 0.15 | 0.15 | 0.15 |
| F | | Ethanol | 3 | 3 | 3 | 3 |
| G | | Sodium Hydroxide | qs pH 7 | qs pH 7 | qs pH 7 | qs pH 7 |

| | | | | |
|---|---|---|---|---|
| Viscosity (mPa · s) | 51589 | 43747 | 19925 | 8548 |
| Texture Transformation | Very | Very | Poor | Very |
| | Good | Good | | Poor |
| Non-Sticky Feeling | Very | Good | Poor | Very |
| | Good | | | Good |
| Non-Oily/Greasy Feeling | Very | Good | Poor | Very |
| | Good | | | Good |
| No-Shiny Skin Aspect | Very | Good | Poor | Very |
| | Good | | | Good |
| Stability | Very | Very | Poor | Very |
| | Good | Good | | Poor |

The properties of the ingredient (a) (cellulose) in Table 1B are shown in Table 2B.

TABLE 2B

| | | Mean Size | WP Oil | WP Water |
|---|---|---|---|---|
| Powder | Shape | (μm) | (ml/100 g) | (ml/100 g) |
| Cellulose | Sphere | 4.0 | 296.0 | 400.8 |
| (Cellulobeads USF, | | | | |
| Daito Kasei) | | | | |

Mean Size: Number-Average Primary Particle Size
WP Oil: Wet Point for Oil
WP Water: Wet Point for Water

### (Wet Point for Oil)

The wet point for oil was determined by the following protocol.
(1) 2 g of the powder component was kneaded with a spatula on a glass plate while adding isononyl isononanoate with a viscosity of 9 cP at 25° C. and a density of 0.853 g/ml.
(2) When the powder component became completely wet and started to form a paste, the weight of the added oil was determined as the weight of the wet point.
(3) The wet point for oil was calculated from the equation: The wet point for oil (ml/100 g)={(the weight of wet point)/2 g}×100/the density of oil.

### (Wet Point for Water)

The wet point for water was determined by the following protocol.
(1) 2 g of the powder component was kneaded with a spatula on a glass plate while adding water with a density of 0.998 g/ml.
(2) When the powder component became completely wet and started to form a paste, the weight of the added water was determined as the weight of the wet point.
(3) The wet point for water was calculated from the equation: The wet point for water (ml/100 g)={(the weight of the wet point)/2 g}×100/the density of water,

### [Evaluations]

### (Viscosity)

The viscosity of each of the compositions according to Examples 1B-2B and Comparative Examples 1B-2B was measured at a room temperature (25° C.) with a B-type viscometer under the conditions of rotor: No. 4, rotation speed: 6 rpm, and measurement time: 1 minute.

The results are shown in Table 1B.

### (Texture Transformation)

Five professional panelists evaluated “texture transformation” during the application of the compositions according to Examples 1B-2B and Comparative Examples 1B-2B. Each panelist took each composition and applied it circularly onto their faces. They evaluated the timing when texture transformation (from gel to liquid) occurred, and graded it from 1 (poor) to 5 (very good), which was then classified in the following 4 categories based on the average of the grade:
Very Good: From 5.0 to 4.0 (texture transformation is clearly felt at the early stage of application)
Good: From 3.9 to 3.0 (texture transformation is felt at the early stage of application)
Poor: From 2.9 to 2.0 (texture transformation is hardly felt at the early stage of application)
Very Poor: From 1.9 to 1.0 (texture transformation is not felt at all at the early stage of application)

The results are shown in Table 1B.

### (Non-Sticky Feeling)

Five professional panelists evaluated the “non-sticky feeling” after application of the compositions according to Examples 1B-2B and Comparative Examples 1B-2B. Each panelist took each composition and applied it onto their faces to evaluate the non-sticky feeling after application, and graded it from 1 (poor) to 5 (very good), which was then classified in the following 4 categories based on the average of the grade:
Very Good: From 5.0 to 4.0 (stickiness is not felt at all after application)
Good: From 3.9 to 3.0 (stickiness is hardly felt after application)
Poor: From 2.9 to 2.0 (stickiness is felt after application)
Very Poor: From 1.9 to 1.0 (stickiness is clearly felt after application)

The results are shown in Table 1B.

### (Non-Oily/Greasy Feeling)

Five professional panelists evaluated the “non-oily/greasy feeling” after application of the compositions according to Examples 1B-2B and Comparative Examples 1B-2B. Each panelist took each composition and applied it onto their faces to evaluate the non-sticky feeling after application, and graded it from 1 (poor) to 5 (very good), which was then classified in the following 4 categories based on the average of the grade:
Very Good: From 5.0 to 4.0 (oiliness/greasiness is not felt at all after application)
Good: From 3.9 to 3.0 (oiliness/greasiness is hardly felt after application)
Poor: From 2.9 to 2.0 (oiliness/greasiness is felt after application)
Very Poor: From 1.9 to 1.0 (oiliness/greasines is clearly felt after application)

The results are shown in Table 1B.

### (No-Shiny Skin Aspect)

Five professional panelists evaluated the “non-shiny skin aspect” after application of the compositions according to Examples 1B-2B and Comparative Examples 1B-2B. Each panelist took each composition and applied it onto their faces to evaluate the non-shiny skin aspect after application, and graded it from 1 (poor) to 5 (very good), which was then classified in the following 4 categories based on the average of the grade:
Very Good: From 5.0 to 4.0 (shininess is not felt at all after application)
Good: From 3.9 to 3.0 (shininess is hardly felt after application)
Poor: From 2.9 to 2.0 (shininess is felt after application)
Very Poor: From 1.9 to 1.0 (shininess is clearly felt after application)

### (Stability)

Each of the compositions according to Examples 1B-2B and Comparative Examples 1B-2B was filled into a glass bottle and was held under temperature changing conditions at 4° C., 25° C., 37° C. and 45° C. for 2 months. Each sample was then investigated for the degree of change (aspect, color, and odor), and evaluated in accordance with the following criteria:
Very Good: Almost the same conditions as at production.
Good: Little changes in aspect, color, and odor were observed.
Poor: Changes in aspect, color, and odor were clearly observed.
Very Poor: Changes in aspect, color, and odor were remarkably noticed.

The results are shown in Table 1B.

### Summary

As is clear from Table 1B, the composition in the form of an O/W emulsion according to the present invention (Examples 1B and 2B) was able to provide excellent cosmetic effects in terms of texture transformation during application; non-sticky feeling, non-oily/greasy feeling, and non-shiny aspect, after application; and stability under temperature changes for 2 months, which could be attributed to a combination of the ingredients (a) and (b) under the presence of the ingredients (c) and (d). Thus, the composition according to the present invention can provide, in particular, an excellent feeling to the touch and stability for a long time period even under temperature changes.

The composition according to Example 2B is more preferable than the composition according to Example 1B, due to the presence of the ingredient (g) (silica particles), in terms of non-sticky feeling, non-oily/greasy feeling and non-shiny aspect, after application.

On the other hand, the compositions according to Comparative Example 1B which did not include the ingredient (a) (cellulose particles) showed inferior texture transformation, stickiness, oiliness/greasiness, and shiny aspect, as well as instability.

The composition according to Comparative Example 2B which did not include the ingredient (b) (acrylates/beheneth-25 methacrylate copolymer) could not be thickened well, and showed inferior texture transformation and instability.

1. A composition, comprising:
(a) at least one cellulose particle;
(b) at least one associative polymer comprising one or more acrylic and/or methacrylic units; and
(c) water.

2. The composition according to claim 1, wherein the particle size of the (a) cellulose particle is 50 μm or less.

3. The composition according to claim 1, wherein the (a) cellulose particle has
a wet point for oil of at least 40 ml/100 g, and
a wet point for water of at least 100 ml/100 g.

4. The composition according to claim 1, wherein the ratio of the wet point for water/the wet point for oil of the (a) cellulose particle is 5 or less.

5. The composition according to claim 1, wherein the (a) cellulose particle is spherical.

6. The composition according to claim 1, wherein the amount of the (a) cellulose particle(s) in the composition is from 0.1% to 20% by weight relative to the total weight of the composition.

7. The composition according to claim 1, wherein the (b) associative polymer comprises one or more (meth)acrylic acid (C₁-C₁₂) alkyl ester units.

8. The composition according to claim 1, wherein the (b) associative polymer comprises one or more units derived from polyoxyalkylenated fatty alcohols.

9. The composition according to claim 1, wherein the amount of the (b) associative polymer(s) in the composition is from 0.01% to 10% by weight relative to the total weight of the composition.

10. The composition according to claim 1, wherein the composition further comprises (d) at least one lipophilic organic UV filter.

11. The composition according to claim 10, wherein the (d) lipophilic organic UV filter is selected from the group consisting of drometrizole trisiloxane, bis(ethylhexyloxyphenol)methoxyphenyltriazine, ethylhexyl triazone, diethylamino hydroxybenzoyl hexyl benzoate, butyl methoxybenzoylmethane, ethylhexyl methoxycinnamate, ethylhexyl salicylate, octocrylene, homosalate, and a mixture thereof.

12. The composition according to claim 10, wherein the amount of the (d) lipophilic organic UV filter(s) in the composition is from 1% to 30% by weight relative to the total weight of the composition.

13. The composition according to claim 1, wherein the composition further comprises (e) at least one oil.

14. The composition according to claim 1, wherein the composition further comprises (f) at least one nonionic surfactant.

15. A cosmetic process for treating a keratin substance, comprising:
applying the composition according to claim 1 to the keratin substance.
